(19) United States
(12) Patent Application Publication
(10) Pub. No.: US 2021/0246485 A1
(43) Pub. Date: Aug. 12, 2021

(54) HISTAMINE MEASUREMENT METHOD AND KIT

(71) Applicant: Kikkoman Corporation, Chiba (JP)

(72) Inventor: Yuko ICHIYANAGI, Chiba (JP)

(73) Assignee: Kikkoman Corporation, Chiba (JP)

(21) Appl. No.: 17/269,081

(22) PCT Filed: Aug. 20, 2019
(86) PCT No.: PCT/JP2019/032370

§ 371 Date: Feb. 17, 2021

(30) Foreign Application Priority Data
Aug. 20, 2018 (JP) 2018-153916

(51) Int. Cl. C12Q 1/32; C07F 5/02; G01N 27/327

(52) U.S. Cl. CPC C12Q 1/32; G01N 27/3277; G01N 27/3271; C07F 5/025

(57) ABSTRACT

An object of the present invention is to reduce error reaction that is not derived from enzyme reaction in a method for measuring histamine using histamine dehydrogenase. The present invention provides a kit for detecting histamine or an electrochemical sensor capable of detecting the oxidation of histamine, comprising (i) histamine dehydrogenase, and (ii-a) boric acid or a salt thereof, and/or a boronic acid or a salt thereof, and/or (ii-b) alkyl sulfate. The present invention also provides a method for detecting histamine using (i) histamine dehydrogenase, and (ii-a) boric acid or a salt thereof, and/or a boronic acid or a salt thereof, or (ii-b) sodium laurel sulfate, or using an electrochemical sensor capable of detecting the oxidation of histamine, comprising these components.

### TECHNICAL FIELD

The present invention relates to a kit for detecting histamine, a sensor capable of detecting histamine, and a method for detecting histamine, etc.

### BACKGROUND ART

Histamine is a chemical mediator of allergic reactions that occur in the body. Therefore, allergy-like poisoning is caused by the ingestion of foods containing histamine accumulated in large amounts. As specific symptoms of allergic reactions caused by histamine, the face and the like become red several minutes to several hours after meals, followed by itchiness, hives, or eczema. In severe cases, hives spread throughout the body and may cause bronchitis or a decrease in blood pressure. In response to this, histamine measurement methods that can measure histamine content readily and rapidly have been strongly demanded for food processing plants, food sanitation monitoring agencies, clinical laboratories, etc.

For example, fluorescent analysis, chromatography based on thin-layer chromatography or paper chromatography, high-performance liquid chromatography (HPLC), antigen-antibody reaction, and enzymatic techniques are known as methods for measuring histamine contents. The present applicant has focused on the enzymatic techniques, which are convenient and highly accurate, and developed so far methods for measuring histamine using histamine dehydrogenase (Patent Literatures 1 and 2).

### CITATION LIST

### Patent Literature

Patent Literature 1: JP Patent Publication (Kokai) No. 2001-157597 A (2001) (JP Patent No. 3926071)

Patent Literature 2: JP Patent Publication (Kokai) No. 2004-129597 A (2004)

### SUMMARY OF INVENTION

### Technical Problem

The present inventor has found that in methods for measuring histamine using histamine dehydrogenase, color development not derived from enzyme reaction (hereinafter, in the present specification, also referred to as “error reaction (erroneous reaction)” or “error color development (erroneous color development)”) may occur and this color development may interfere with the detection of histamine, particularly, in a low-concentration range. Thus, an object of the present invention is to reduce this error reaction.

### Solution to Problem

The present inventor has completed the invention of the present application by finding that boric acid or a salt thereof, and/or a boronic acid or a salt thereof, and/or alkyl sulfate is capable of reducing the error reaction. Accordingly, the present invention encompasses the following aspects.

(1) A kit for detecting histamine, comprising

(i) histamine dehydrogenase, and
(ii-a) boric acid or a salt thereof, and/or a boronic acid represented by the following formula (I) or (II):

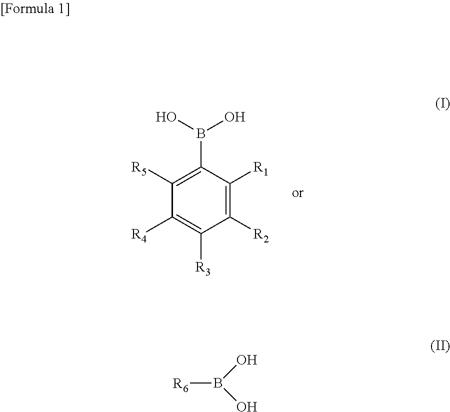

wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

(2) An electrochemical sensor capable of detecting the oxidation of histamine by histamine dehydrogenase, comprising

(i) histamine dehydrogenase, and
(ii-a) boric acid or a salt thereof, and/or a boronic acid represented by the following formula (I) or (II):

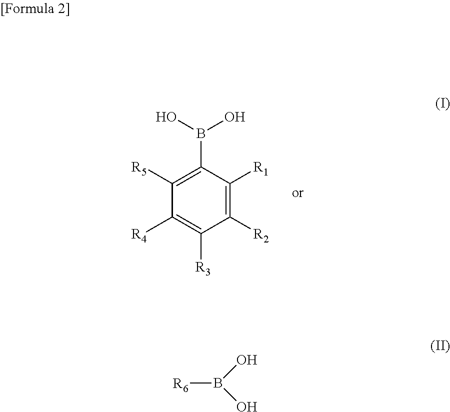

wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

(3) The kit according to (1) or the sensor according to (2), further comprising a mediator.

(4) The kit or the sensor according to (3), wherein the mediator is selected from the group consisting of 1-methoxy PMS (1-methoxy-5-ethylphenazinium ethyl sulfate), PMS (phenazinium methyl sulfate), PES (phenazinium ethyl sulfate) and 1-methoxy PES (1-methoxy-5-ethylphenazinium ethyl sulfate).

(5) The kit or the sensor according to any of (1) to (4), further comprising a coloring reagent that develops a color when histamine is oxidized by the histamine dehydrogenase.

(6) the kit or the sensor according to (5), wherein the coloring reagent is tetrazolium salt.

(7) The kit or the sensor according to any of (1) to (6), further comprising a buffer comprising a compound having a zwitterion and having no carboxy group, the buffer being selected from the group consisting of BES, MOPS, TES, HEPES, EPPS, TAPS, CHES, CAPS, TAPSO, POPSO, HEPPSO, ACES, Bis-Tris, MES, MOPSO, and PIPES, or Tris (tris(hydroxymethyl)aminomethane) or a carbonate buffer.

(8) The kit or the sensor according to (7), wherein the buffer is a buffer comprising a compound having a zwitterion and having a sulfo group, the buffer being selected from the group consisting of BES, MOPS, TES, HEPES, EPPS, TAPS, CHES, CAPS, TAPSO, POPSO, HEPPSO, ACES, MES, MOPSO, and PIPES.

(9) The kit or the sensor according to (8), wherein the buffer is a buffer comprising a compound having a zwitterion, having a sulfo group, and having a hydroxy group at position 2, the buffer being selected from the group consisting of TAPSO, POPSO, HEPPSO, and MOPSO.

(10) The kit or the sensor according to any of (1) to (9), wherein the kit or the sensor comprises the boric acid or the salt thereof, and/or the boronic acid or the salt thereof such that a final concentration of said boric acid or the salt thereof, and/or the boronic acid or the salt thereof at the time of measurement is 120 mM or lower.

(11) The kit or the sensor according to any of (1) to (10), wherein the alkyl sulfate is sodium lauryl sulfate.

(12) The kit or the sensor according to any of (1) to (11), wherein
the kit or the sensor comprises a sample collection part and a reaction part, and
the reaction part comprises the histamine dehydrogenase and the boric acid or the salt thereof, and/or the boronic acid or the salt thereof.

(13) A method for detecting histamine, comprising using (i) histamine dehydrogenase, and (ii-a) boric acid or a salt thereof, and/or a boronic acid represented by the following formula (I) or (II):

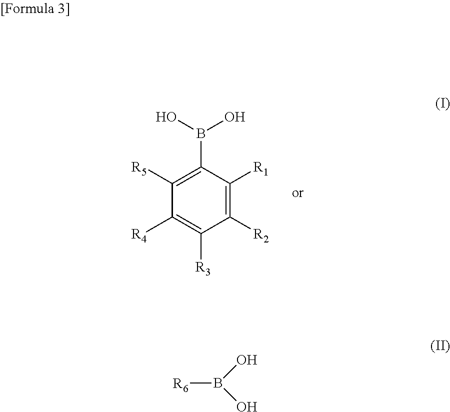

wherein R₁ to R5 are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

(14) A method for detecting histamine, comprising
using an electrochemical sensor capable of detecting the oxidation of histamine by histamine dehydrogenase, wherein the sensor comprises
   (i) histamine dehydrogenase, and
   (ii-a) bode acid or a salt thereof, and/or a boronic acid represented by the following formula (I) or (II):

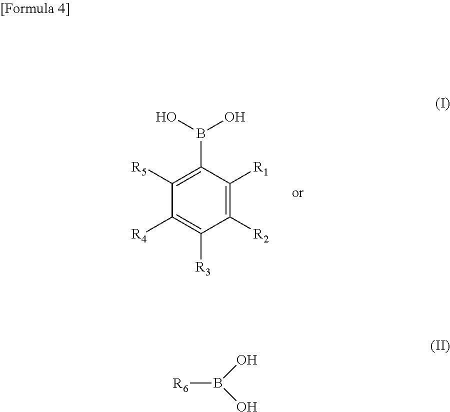

wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

(15) The method according to (13) or (14), further comprising using a mediator,

(16) The method according to (15), wherein the mediator is selected from the group consisting of 1-methoxy PMS, PMS, PES and 1-methoxy PES.

(17) The method according to any of (13) to (16), further comprising using a coloring reagent that develops a color when histamine is oxidized by the histamine dehydrogenase.

(18) The method according to (17), wherein the coloring reagent is tetrazolium salt.

(19) The method according to any of (13) to (18), further comprising using a buffer comprising a compound having a zwitterion and having no carboxy group, the buffer being selected from the group consisting of BES, MOPS, TES, HEPES, EPPS, TAPS, CHES, CAPS, TAPSO, POPSO, HEPPSO, ACES, Bis-Tris, MES, MOPSO, and PIPES, or Tris or a carbonate buffer.

(20) The method according to (19), wherein the buffer is a buffer comprising a compound having a zwitterion and having a sulfo group, the buffer being selected from the group consisting of BES, MOPS, TES, HEPES, EPPS, TAPS, CHES, CAPS, TAPSO, POPSO, HEPPSO, ACES, MES, MOPSO, and PIPES.

(21) The method according to (20), wherein the buffer is a buffer comprising a compound baying a zwitterion, having a sulfo group, and having hydroxy group at position 2, the buffer being selected from the group consisting of TAPSO, POPSO, HEPPSO, and MOPSO.

(22) The method according to any of (13) to (21), wherein the boric acid or the salt thereof, and/or the boronic acid or the salt thereof is used at a final concentration of 120 mM or lower at the time of measurement.

(23) The method according to any of (13) to (22), wherein the alkyl sulfate is sodium lauryl sulfate.

The present specification encompasses the contents disclosed in Japanese Patent Application No. 2018-153916 on which the priority of the present application is based.

### Advantageous Effects of Invention

Accordingly to the present invention, error reaction not derived from enzyme reaction can be reduced in methods for measuring histamine using histamine dehydrogenase. As a result, histamine can be detected, particularly, even in low-concentration range.

### BRIEF DESCRIPTION OF DRAWINGS

FIG. 1-1 shows results of measuring histamine contents using various coloring reagents. The results shown were obtained using WST-4 (A), WST-5 (B), WST-8 (C), INT (D), NBT (E), and XTT (F) as the coloring reagents.

FIG. 1-2 shows the degree of error reaction in the case of using EDTA (2Na) in a system free from histamine and histamine dehydrogenase.

FIG. 2 shows the degree of error reaction in the case of using various buffers. The results shown were obtained using EDTA (2Na) (1), BES (2), MOPS (3), TES (4), HEPES (5), TAPSO (6), POPSO (7), HEPPSO (8), EPPS (9), Tricine (10), Bicine (11), TAPS (12), CHES (13), and CAPS (14).

FIG. 3 shows the degree of error reaction in the case of using various buffers. The results shown were obtained using EDTA (2Na) (1), carbonic acid (2), boric acid (3), and Tris (4).

FIG. 4 shows the degree of error reaction in a HEPPSO buffer (A) or a solution containing a HEPPSO buffer and boric acid (B).

FIG. 5 shows the degree of error reaction in the case of using various boronic acids. The results shown were obtained using a control without the addition of boronic acid (1), phenylboronic acid (2), 4-chlorophenylboronic acid (3), 4-fluorophenylboronic acid (4), butylboronic acid (5), and 3-[(tert-butoxycarbonyl)amino]phenylboronic acid (6).

FIG. 6 shows the degree of error reaction in the case of using boric acid having each concentration from 0 mM to 100 mM.

FIG. 7 shows the degree of error reaction in the case of using SDS having each concentration from 0% to 1%.

FIG. 8 shows results of measuring histamine concentrations using 25 mM or 100 mM boric acid.

FIG. 9 shows a histamine calibration curve obtained by cyclic voltammetry using a boric acid solution or an EDTA solution.

FIG. 10 shows a histamine calibration curve obtained by chronoamperometry using a 0.1 M HEPPSO solution (pH 8.5) or a 0.1 M HEPPSO/0.1 M boric acid solution (pH 8.5).

### DESCRIPTION OF EMBODIMENTS

### 1. Kit for Detecting Histamine or Sensor

In one aspect, the present invention relates to a kit for detecting histamine, comprising (i) histamine dehydrogenase, and (ii-a) boric acid or a salt thereof, and/or a boronic acid or a salt thereof, and/or (ii-b) alkyl sulfate. In the present specification, histamine also includes a salt thereof.

In one embodiment, the kit of the present invention is used in the detection of histamine in a sample which may contain histamine. Examples of the sample which may contain histamine include liquid and solid foods (e.g., fish meat, livestock meat, cheese, soy sauce, fish sauce and wine), body fluids such as urine and plasma, biological materials, and living tissues.

In the present specification, the “histamine dehydrogenase” is an enzyme that is classified into oxidoreductase and catalyzes the oxidation of histamine through the following reaction:

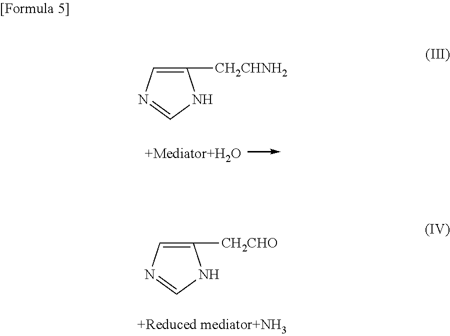

In the reaction formula, the compound of the formula (III) is histamine, and the compound of the formula (IV) is 4-imidazolylacetaldehyde. In one embodiment, the mediator is a mediator described in the present specification, for example, PMS (phenazinium methyl sulfate). In this case, the reduced mediator is reduced PMS (PMSH₂).

From the viewpoint of measurement accuracy, preferably, the histamine dehydrogenase specifically acts on histamine. In other words, it is preferred that the histamine dehydrogenase specifically acts on histamine without acing on other amines or acts only weakly on other amines. It is particularly preferred that the histamine dehydrogenase acts on histamine without acting on cadaverine and putrescine. The histamine dehydrogenase is preferably derived from a bacterium, for example, a bacterium belonging to the genus Rhizobium. The histamine dehydrogenase may be obtained by extracting and/or purifying a naturally derived histamine dehydrogenase or may be produced by a genetic engineering approach known in the art based on genetic information on an organism. A specific method for producing the histamine dehydrogenase is known to those skilled in the art. For example, a method described in JP Patent Publication (Kokai) No. 2001-157597 A (2001) can be used. Specific examples of the histamine dehydrogenase that can be used in the present invention include histamine dehydrogenase described in JP Patent. Publication (Kokai) No. 2001-157597 A (2001). In one embodiment, in the kit of the present invention, the final concentration of the histamine dehydrogenase at the time of measurement or storage, preferably at the time of measurement, is not limited and can be 1 mU/assay to 20 U/assay, preferably 5 mU/assay to 2 U/assay, more preferably 10 mU/assay to 0.5 mU/assay, further preferably 25 mU/assay to 0.25 U/assay (wherein 1 U is defined as the amount of the enzyme that produces 1 μmol 4-imidazolylacetaldehyde at 37° C. at pH 9.0 for 1 minute). The concentration of the histamine dehydrogenase described above is given for illustrative purposes and can be appropriately adjusted according to the reaction time.

In the present specification, the “boric acid” means an oxo acid of boron represented by the chemical formula B(OH)₃. In the present specification, a “boronic acid” is obtained by the substitution of a hydroxy group in boric acid and may refer to a compound represented by the following formula (I) or (II):

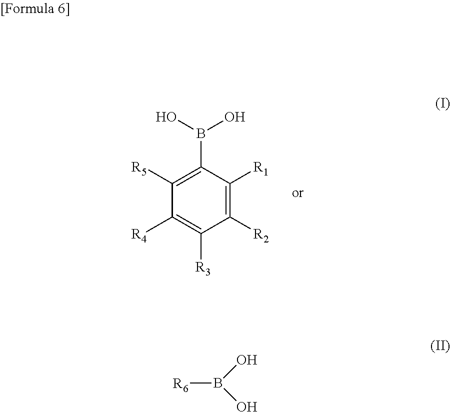

wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ (e.g., C₁ to C₈, C₁ to C₆, or C₁ to C₄) alkyl group, alkenyl group, or alkynyl group, and
R₆ is selected from the group consisting of a C₁ to C₁₀ (e.g., C₁ to C₈, C₁ to C₆, or C₁ to C₄) alkyl group, alkenyl group, or alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ (e.g., C₁ to C₈, C₁ to C₆, or C₁ to C₄) alkyl group, alkenyl group, or alkynyl group. In the compound of the formula (I), at least one of R₁ to R₅ is a halogen group, preferably fluorine or chlorine, or a tert-butoxycarbonylamino group. In the present specification, examples of the boronic acid include, but are not limited to, butylboronic acid, 4-chlorophenylboronic acid, 4-fluorophenylboronic acid, and 3-[(tert-butoxycarbonyl)amino]phenylboronic acid.

In the present specification, the “salt” refers to a salt of an active compound prepared using a base or an acid based on a particular substituent (e.g., a hydroxy group) of a compound. The salt can be classified into a base-addition salt and an acid-addition salt depending on the base or the acid used.

Examples of the “base-addition salt” include: alkali metal salts such as sodium salt and potassium salt; alkaline earth metal salts such as calcium salt and magnesium salt; aliphatic amine salts such as trimethylamine salt, ethanolamine salt, and procaine salt; aralkylamine salts such as N,N-dibenzylethylenediamine; heterocyclic aromatic amine salts such as pyridine salt; basic amino acid salts such as arginine salt; quaternary ammonium salts such as tetramethylammonium salt, tetraethyl ammonium salt, and benzyl trimethylammonium salt; and ammonium salts.

Examples of the “acid-addition salt” include: inorganic acid salts such as hydrochloride salt, sulfate salt, nitrate salt, phosphate salt, carbonate salt, bicarbonate salt, and perchlorate salt; organic acid salts such as acetate salt, propionate salt, lactate salt, maleate salt, fumarate salt, tartrate salt, malate salt, citrate salt, and ascorbate salt; sultanates such as methanesulfonate salt, isethionate salt, benzenesulfonate salt, and p-toluenesulfonate salt; and acidic amino acid (salts) such as aspartate salt and glutamate salt.

In the kit of the present invention, the concentration of the boric acid or the salt thereof, and/or the boronic acid or the salt thereof is not limited as long as the concentration is capable of reducing error reaction not derived from the oxidation of histamine by histamine dehydrogenase (in the present specification, also simply referred to as “error reaction”). Such a concentration can be readily determined by those skilled in the art with reference to the description of the present specification. As an example, the final concentration of the boric acid or the salt thereof, and/or the boronic acid or the salt thereof at the time of measurement or storage, preferably at the time of measurement, can be 5 mM or higher, 10 mM or higher, 20 mM or higher, 25 mM or higher, 50 mM or higher, 80 mM or higher, or 100 mM or higher, and is concentration of, for example, 1000 mM or lower, 500 mM or lower, 400 mM or lower, 300 mM or lower, 200 mM or lower, 150 mM or lower, or 120 mM or lower. The final concentration at the time of measurement or storage can be, for example, 5 mM to 1000 mM, 25 mM to 300 mM, or 50 mM to 200 mM.

In the kit of the present invention, the type of the alkyl sulfite is not limited as long as the type is capable of reducing error reaction and the alkyl sulfate can be one or more members selected from the group consisting of, for example, alkyl sulfuric acid ester salts such as sodium lauryl sulfate, potassium lauryl sulfate, sodium stearyl sulfate, sodium stearyl sulfate triethanolamine lauryl sulfate, and ammonium lauryl sulfate; and polyoxyethylene alkyl sulfates such as sodium laureth sulfate and triethanolamine laureth sulfate. The alkyl sulfate is, for example, sodium lauryl sulfate.

In the kit of the present invention, the concentration of the alkyl sulfate is not limited as long as the concentration is capable of reducing error reaction. Such concentration can be readily determined by those skilled in the art with reference to the description of the present specification. As an example, the final concentration of the alkyl sulfate at the time of measurement or storage, preferably at the time of measurement, is 0.01% or higher, 0.05% or higher, preferably 0.1% or higher, 0.5% or higher, or 1% or higher, and 10% or lower, 5% or lower, or 2% or lower. The final concentration is a concentration of, for example, approximately 0.05% to 2% or approximately 0.1 to 1%.

The degree of error reaction may be influenced by conditions such as the type of the sample used, in addition to the active ingredient described above such as boric acid or a salt thereof, and/or a boronic acid or a salt thereof, and/or alkyl sulfate. The degree of reduction in error reaction can be evaluated more accurately by adding a control sample free from the active ingredient to a test system, and comparing the sample with the control sample. The degree of reduction in error reaction can be evaluated, for example, by visual confirmation or by the comparison of colors digitized using software such as illustrator CS2 (manufactured by Adobe Inc.), as described in Examples.

In one embodiment, the kit of the present invention further comprises a mediator. In the present specification, the “mediator” refers to a molecule that facilitates oxidoreduction reaction catalyzed by histamine dehydrogenase, for example, through action as a cofactor. The mediator is preferably a substance that promotes electron transfer from a substrate to a coloring reagent or an electrode. An appropriate mediator for a reaction system can be readily selected by those skilled in the art and examples of the mediator include, but are not limited to, 1-methoxy PMS (1-methoxy-5-methylphenazinium methyl, sulfate), PMS (phenazinium methyl sulfate), PES (phenazinium ethyl sulfate), 1-methoxy PES (1-methoxy-5-ethylphenazinium ethyl sulfate), benzoquinone and derivatives thereof, ferricyanide (potassium or sodium salt), ferrocene and derivatives thereof, dichlorophenolindophenol, naphthoquinone and derivatives thereof, phenanthrolinequinone and derivatives thereof, phenanthrenequinone and derivatives thereof, anthraquinone and derivatives thereof, ruthenium salt, and ruthenium complexes and the mediator is preferably 1-methoxy PMS, PMS, PES or 1-methoxy PES, more preferably 1-methoxy PMS. In one embodiment, the kit of the present invention comprises the mediator having a final concentration of 1 μM or higher, 10 μM or higher, 20 μM or higher, 25 μM or higher, 30 μM or higher, or 35 μM or higher, and 80 μM or lower, 70 μM or lower, 60 μM or lower, 50 μM or lower, or 45 μM or lower, for example, 1 μM to 80 μM, 35 μM to 45 μM or approximately 42 μM, at the time of measurement or storage.

In one embodiment, the kit of the present invention further comprises a coloring reagent. The coloring reagent preferably develops a color when histamine is oxidized by histamine dehydrogenase and the presence of histamine can be conveniently detected by observing such color development. Examples of the coloring reagent include tetrazolium salt, for example, WST-4 (2-benzothiazolyl-3-(4-carboxy-2-methoxyphenyl)-1)-[4-(2-sulfoethylcarbamoyl)phenyl]-2H-tetrazolium), WST-5 (2,2′- dibenzothiazolyl-5,5′-bis[4-di(2-sulfoethyl)carbamoylphenyl]-3,3′-(3,3′-dimethoxy-biphenylene)ditetrazolium, disodium salt), WST-8 (2-(2-methoxy-4-nitrophenyl)-3-(4-nitrophenyl)-5-(2,4-disulfophenyl)-2H-tetrazolium), NBT (3,3′-[3,3′-dimethoxy-(1,1′-biphenyl)-4,4′-diyl]-bis[2-(4-nitrophenyl)-5-phenyl-2H-tetrazolium chloride]), INT (2-(4-iodophenyl)- 3-(4-nitrophenyl)-5-phenyl-2H-tetrazolium chloride), and XTT (2,3-bis(2-methoxy-4-nitro-5-sulfophenyl)-5-[(phenylamino)carbonyl]-2H-tetrazolium hydroxide). The tetrazolium salt such as WST-4, WST-5, WST-8, NBT, INT, and XTT generates a formazan dye when reduced. The detection of this dye enables histamine to be detected. In one embodiment, the chromogenic reaction of the coloring reagent upon oxidation of histamine is promoted via the mediator. In one embodiment, the kit of the present invention comprises the coloring reagent having a final concentration of 0.1 mM or higher, 0.2 mM or higher, 0.3 mM or higher, 0.4 mM or higher, or 0.5 mM or higher, and 10 mM or lower, 5 mM or lower, or 2 mM or lower, for example, 0.1 mM to 10 mM, 0.5 mM to 2 mM or 1.1 mM, at the time of measurement or storage.

In one embodiment, the kit of the present invention further comprises a buffer. Examples of the buffer include a buffer that is selected from the group consisting of BES, MOPS, TES, HEPES, EPPS, TAPS, CHES, CAPS, TAPSO, POPSO, HEPPSO, ACES, Bis-Tris, MES, MOPSO, and PIPES, and contains a compound having a zwitterion and having no carboxy group, and Tris and a carbonate buffer. The buffer containing a compound having a zwitterion and having no carboxy group is preferably selected from the group consisting of BES, MOPS, TES, HEPES, EPPS, TAPS, CHES, CAPS, TAPSO, POPSO, and HEPPSO, and more preferably selected from the group consisting of HEPES, TAPSO, POPSO, HEPPSO, and EPPS. In one embodiment, the buffer is a buffer selected from the group consisting of BES, MOPS, TES, HEPES, EPPS, TAPS, CHES, CAPS, TAPSO, POPSO, HEPPSO, ACES, MES, MOPSO, and PIPES, preferably the group consisting of BES, MOPS, TES, HEPES, EPPS, TAPS, CHES, CAPS, TAPSO, POPSO, and HEPPSO, and contains a compound having a zwitterion and having a sulfo group, or Tris or a carbonate buffer. In one embodiment, the buffer is a buffer that is selected from the group consisting of TAPSO, POPSO, HEPPSO, and MOPSO, preferably the group consisting of TAPSO, POPSO, and HEPPSO, and contains a compound having a zwitterion, having a sulfo group, and having a hydroxy group at position 2, or Tris or a carbonate buffer. The pH of the buffer is preferably on the order of 6.0 to 11.0 and can be more preferably 7.0 or higher, 8.0 or higher or 8.5 or higher, and 10.0 or lower or 9.5 or lower, for example, approximately 8.5 to 9.5. As for the concentration of the buffer, the final concentration at the time of measurement or storage, preferably at the time of measurement, can be, for example, 1 mM or higher, 10 mM or higher, or 50 mM or higher, and 300 mM or lower, 200 mM or lower, or 150 mM or lower, for example, 1 mM to 300 mM, 50 mM to 150 mM, or approximately 100 mM.

In one embodiment, the kit of the present invention further comprises an extracting liquid for extracting histamine from a sample which may contain histamine. As the extracting liquid, extracting liquids known in the art can be used. For example, trichloroacetic acid, methanol, or a neutral phosphate buffer (JP Patent Publication (Kokai) No. 2001-099803 A (2001)), or an extracting liquid containing a chelating agent (JP Patent Publication (Kokai) No. 2004-129597 A (20041) can be used, and water or various buffers can also be used. The kit of the present invention may comprise an additional component (e.g., sugar (lactose, maltose, galactose, sucrose, glucose, trehalose, etc.), starch (including soluble starch), and dextrin (including branched dextrin, cyclodextrin, and highly branched cyclic dextrin (cluster dextrin))) and/or an instruction for use.

In one embodiment, the kit of the present invention comprises a sample collection part and a reaction part. The sample collection part is not particularly limited as long as the sample collection part is capable of collecting a sample which may contain histamine and examples thereof include cotton-tipped swabs, sponges, porous plastics, filter papers, nonwoven fabrics, and droppers. The sample collection part is preferably in the shape of, for example, a rod, particularly preferably the shape of a rod having, a fibrous or spongy wiping portion, for example, a cotton-tipped swab, from the viewpoint of convenient sample collection.

The reaction part is a site where reaction occurs when histamine is present in the sample collected by the sample collection part. In one embodiment, the histamine dehydrogenase and the boric acid or the salt thereof, and/or the boronic acid or the salt thereof, and/or the alkyl sulfate described in the present specification are included in the sample collection part or the reaction part in the kit of the present invention in a form included in a solution such as the buffer described above or in a form included as a freeze-dried product. The reaction part is preferably a transparent container through which the presence or absence of color development can be visually observed to detect histamine.

In one embodiment, the kit of the present invention comprises an extraction part in addition to the sample collection part and the reaction part. The extraction part is a part (site) where histamine is extracted into an extracting liquid when this histamine is present in the sample collected by the sample collection part. The histamine dehydrogenase and the boric acid or the salt thereof, and/or the boronic acid or the salt thereof, and/or the alkyl sulfate described in the present specification may be included in the extraction part in the kit of the present invention in a form included in a solution such as the buffer described above or in a form included as a freeze-dried product. The sample containing histamine extracted by the extraction part can be transferred to the reaction part where reaction is then performed.

In the present specification, the “detection” of histamine includes the detection of the presence or absence of histamine as well as the quantification of histamine. The quantification can be performed based on the degree of color development in the case of using a chromogenic substrate or based on a signal (current value) in the case of using an electrochemical sensor. The quantification can be performed, preferably based on a calibration curve, for example, by using a plurality of, for example, 2 or more, preferably 3 or more, 4 or more, or 5 or more samples containing known concentrations of histamine, and comparing the sample of concern with these (known) samples.

In one embodiment, the kit of the present invention further comprises an electrochemical sensor capable of detecting the oxidation of histamine by histamine dehydrogenase. In this embodiment, the kit of the present invention may be a kit that does not comprise a coloring reagent.

The electrochemical sensor basically comprises an electrode and a circuit system. The electrode may be a three-electrode system (working electrode, reference electrode and counter electrode) and may preferably be two electrodes (working electrode and reference electrode). The type of electrode is not limited and for example, platinum, gold silver, or carbon such as glassy carbon can be used in the working electrode. A hydrogen electrode, a saturated calomel electrode, silver-silver chloride, a silver electrode, and a palladium-hydrogen electrode can be used as the reference electrode.

The electrochemical sensor is capable of measuring change in current caused by oxidoreduction reaction through which histamine is oxidized by the catalytic effect of histamine dehydrogenase to produce 4-imidazolylacetaldehyde. In this respect, the kit preferably comprises a mediator winch promotes electron transfer to an electrode.

In one embodiment, the histamine dehydrogenase is preferably fixed on the working electrode of the electrochemical sensor via a covalent bond.

The method for detection using the electrochemical sensor is not limited and can be, for example, cyclic voltammetry or chronoamperometry and such a method can be performed, for example, as described in Examples.

A commercially available product may be used as the electrochemical sensor. For example, SCREEN-PRINTEDELECTRODES (manufactured by Metrohm DropSens, DRP-110) can be used as the electrodes. A dedicated connector (manufactured by Metrohm DropSens, DRP-CAC) can be used as the circuit. ALS electrochemical analyzer 814D (manufactured by BAS Inc.) can be used for the measurement of change in current.

In one embodiment, the kit of the present invention comprises histamine dehydrogenase, boric acid or a salt thereof, and/or a boronic acid or a salt thereof, a mediator, and a coloring reagent. In another embodiment, the kit of the present invention comprises histamine dehydrogenase, boric acid or a salt thereof, and/or a boronic acid or a salt thereof, a mediator, and an electrochemical sensor.

In one aspect, the present invention relates to an electrochemical sensor capable of detecting the oxidation of histamine by histamine dehydrogenase, comprising (i) histamine dehydrogenase, and (ii-a) boric acid or a salt thereof, and/or a boronic acid or a salt thereof, and/or (ii-b) alkyl sulfate. The configuration of the electrochemical sensor is as described above. In the electrochemical sensor according to the present aspect, detailed histamine, histamine dehydrogenase, boric acid, boronic acid, and alkyl sulfate are as described above with regard to the kit. In one embodiment, the electrochemical sensor of the present invention further comprises one or more, preferably all, of a mediator, a buffer and an extracting liquid. These components are also as described above with regard to the kit.

### 2. Method for Detecting Histamine

In one aspect, the present invention relates to a method for detecting histamine, comprising using (i) histamine dehydrogenase, and (ii-a) boric acid or a salt thereof, and/or a boronic acid or a salt thereof, and/or (ii-b) alkyl sulfate. This method may be performed using the kit described in the preceding section “1. Kit for detecting histamine or sensor”. In another aspect, the present invention relates to a method for detecting histamine comprising using an electrochemical sensor capable of detecting the oxidation of histamine by histamine dehydrogenase, the electrochemical sensor comprising (i) histamine dehydrogenase, and (ii-a) boric acid or a salt thereof, and/or a boronic acid or a salt thereof, and/or (ii-b) alkyl sulfate. This method may be performed using the sensor described in the preceding section “1. Kit for detecting histamine or sensor”.

The method for detecting histamine according to the present invention comprises the steps of: oxidizing histamine with histamine dehydrogenase (hereinafter, also referred to as an “oxidation step”); and detecting the oxidation of histamine with histamine dehydrogenase (hereinafter, also referred to as a “detection step”).

The oxidation step can be performed by a method known to those skilled in the art. The oxidation step can be performed, for example, by mixing a sample which may contain histamine with a solution containing the histamine dehydrogenase described in the present specification.

The detection step can also be performed by a method known to those skilled in the art. The detection of the oxidation of histamine can be performed, for example, using a coloring reagent or an electrochemical sensor and optionally further using a mediator. The presence or absence of histamine can be detected, or the amount thereof can be measured, based on the presence or absence or the degree of the oxidation in the oxidation step.

The method for detecting histamine according to the present invention optionally comprises a sample collection step and/or a histamine extraction step before the oxidation step, in addition to the oxidation step and the detection step.

In the sample collection step, a sample is collected in a manner suitable for the method of the present invention from a sample in which histamine is to be measured. The sample collection can be performed, for example, by bringing a sample collection part such as a cotton-tipped swab, a sponge, a porous plastic, a filter paper, a nonwoven fabric, or a dropper, particularly preferably a sample collection part in the shape of a rod having a fibrous or spongy wiping portion, for example, a cotton-tipped swab, into contact with a sample in which histamine is to be measured.

In the histamine extraction step, the subsequent oxidation step and detection step are facilitated by extracting histamine from the collected sample. The histamine extraction step can be performed by mixing the sample in which histamine is to be measured (e.g., the collection part that has collected the sample when the method of the present invention comprises the sample collection step) with a histamine-extracting liquid. A histamine-extracting liquid known in the art can be used and for example, trichloroacetic acid, methanol, or a neutral phosphate buffer (JP Patent Publication (Kokai) No. 2001-099803 A (2001)), or an extracting liquid containing a chelating agent (JP Patent Publication (Kokai) No. 2004-129597 A (2004)), water or various buffers can also be used.

### EXAMPLES

Examples given below intend only illustration and do not intend to limit the technical scope of the present invention by any means. Unless otherwise specified, reagents are commercially available, or are obtained or prepared according to approaches commonly used in the art or procedures of literatures known in the art.

### Example 1: Study on Coloring Reagent

An aliquot of 0.2 mL of a histamine measurement reagent containing 2% trehalose, a coloring reagent (1.08 mM WST-4, WST-5, WST-8, INT, NBT, or XTT), 41.5 μM 1-methoxy PMS, and 0.128 U histamine dehydrogenase (prepared according to JP Patent Publication (Kokai) No. 2001-157597 A (2001)) was placed into each measurement tube and freeze-dried. Histamine solutions having various known concentrations were prepared for calibration curve preparation, and 0.1 mL aliquots were then added to cotton-tipped swabs. As for samples, 0.1 mL of a drip of a tuna strip (Saku block tuna) was added to each cotton-tipped swab, or the surface of a tuna strip (Saku block tuna) was swabbed.

Each cotton-tipped swab was pushed into a container loaded with 0.4 mL of EDTA (2Na) with pH adjusted to 9.0 as an extracting liquid, which was further shaken down to the measurement tube where the extracting liquid was then reacted with the histamine measurement reagent.

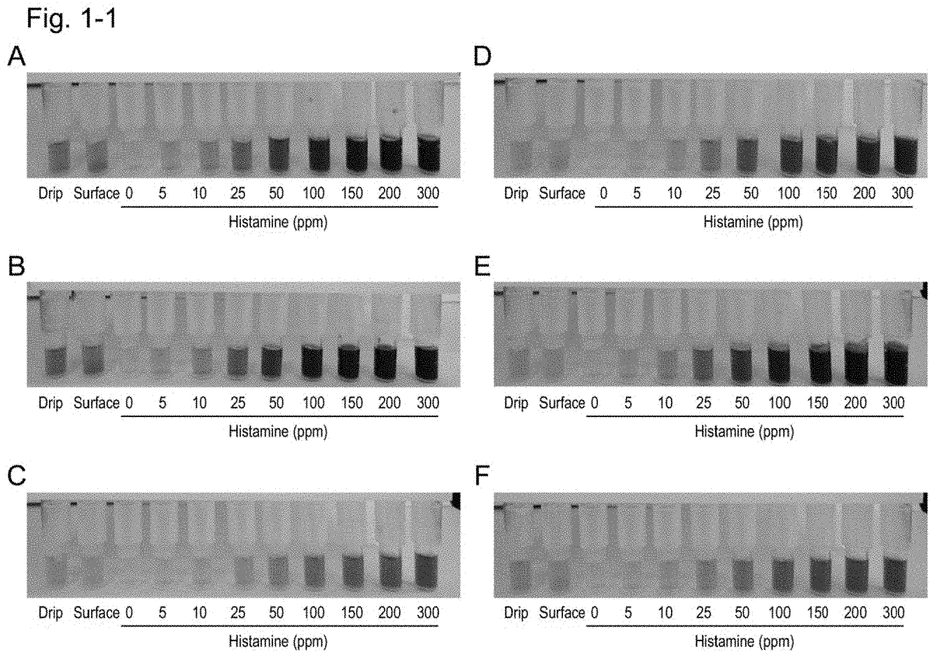

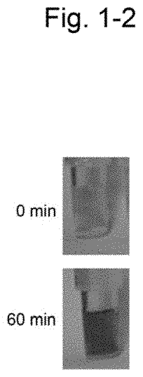

The results are shown in FIG. 1-1. As shown in FIG. 1-1, it was found that histamine contained in a sample can be visually detected using any of the coloring reagents WST-4, WST-5, WST-8, NBT, INT, and XTT and its approximate concentration can also be determined.

For the purpose of also confirming the visual determination as shown in FIG. 1-1 with numerical data, colors were digitized by the following method.

Photographs of the measurement reagent tubes after color development were taken using STYLUS TG-4 Tough (manufactured by Olympus Corp.). Then, colors at or near the center of the images of the measurement tubes were converted to numerical values of CMYK (cyan, magenta, yellow, and black). Specifically, Illustrator CS2 (manufactured by Adobe Inc.) was used, and the center or its neighborhood of the measurement tube photographs was selected with the Eye-Dropper Tool in the Toolbox. The colors were digitized by confirming numerical values (%) through conversion into CMYK color codes in the Color Palette. For digitization, the “value of K” was used in all cases. The results are shown in Table 1.

TABLE 1

| | | WST-4 | WST-5 | WST-8 | NBT | INT | XTT |
|---|---|---|---|---|---|---|---|
| Drip | | 33.3 | 42.8 | 20.0 | 20.0 | 25.5 | 26.7 |
| Swab | | 30.6 | 38.0 | 14.5 | 19.6 | 19.2 | 20.8 |
| Histamine | 0 | 9.0 | 12.6 | 4.7 | 4.7 | 6.7 | 6.7 |
| concen- | 5 | 15.3 | 16.5 | 8.6 | 7.5 | 12.2 | 10.2 |
| tration | 10 | 24.7 | 23.9 | 10.6 | 12.6 | 19.6 | 15.7 |
| (ppm) | 25 | 40.0 | 46.7 | 16.5 | 27.1 | 38.4 | 25.1 |
| | 50 | 56.5 | 64.3 | 21.6 | 40.4 | 54.9 | 29.4 |
| | 100 | 67.1 | 71.4 | 28.6 | 51.8 | 68.6 | 34.5 |
| | 150 | 71.8 | 72.9 | 32.2 | 57.7 | 72.2 | 36.9 |
| | 200 | 71.8 | 72.9 | 33.7 | 63.9 | 74.1 | 37.7 |
| | 300 | 72.9 | 72.6 | 38.0 | 66.7 | 74.9 | 39.2 |

Also, a test was conducted in a system free from histamine and histamine dehydrogenase as described below. Specifically, EDTA (2Na) was adjusted to 0.1 M and pH 8.5, and 0.4 of the resultant was added to a freeze-dried product of 0.2 mL of a reagent containing 2% trehalose, 1.08 mM NBT, and 41.5 μM 1-methoxy PMS. After mixing, the mixture was left at room temperature for 60 minutes, and the degree of color development was confirmed.

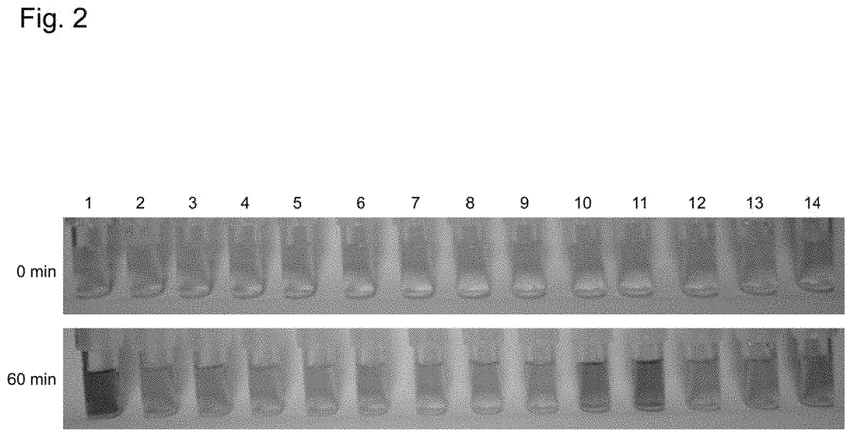

The results are shown in FIG. 1-2. For the purpose of also confirming the visual determination as shown in FIG. 1-2 with numerical data, colors were digitized in the same manner as above. For digitization, the “value of K” was used in all cases and the difference in color between before and after leaving the samples for 60 minutes was 27.45. As described above, a certain amount of color development (error reaction) was confirmed even in the case of using the system free from histamine and histamine dehydrogenase.

### Example 2: Study on Buffer-1

In Example 1, color development was observed even in histamine-free samples and, therefore, buffers were studied in order to inhibit or reduce this error reaction.

EDTA (2Na), BES, MOPS, TES, HEPES, TAPSO, POPSO, HEPPSO, EPPS, Tricine, Bicine, TAPS, CHES, and CAPS were all adjusted to 0.1 M and pH 8.5, and 0.4 mL of the resultant was added to a freeze-dried product of 0.2 mL of a reagent containing 2% trehalose, 1.08 mM NBT, and 41.5 μM 1-methoxy PMS. After mixing, the mixture was left at room temperature for 60 minutes, and the degree of color development was confirmed.

The results are shown in FIG. 2. The results of experimental system No. 1 are the same as those of Example 1. For the purpose of also confirming the visual determination as shown in FIG. 2 with numerical data, colors were digitized in the same manner as in Example 1. For digitization, the “value of K” was used in all cases. The difference in color between before and after leaving the samples for 60 minutes is shown in Table 2 below.

TABLE 2

| Experimental | | |
|---|---|---|
| system No. | Buffer | Color change |
| 1 | EDTA | 27.45 |
| 2 | BES | 1.96 |
| 3 | MOPS | 3.52 |
| 4 | TES | 0.4 |
| 5 | HEPES | 0 |
| 6 | TAPSO | 0.39 |
| 7 | POPSO | 0.39 |
| 8 | HEPPSO | 0 |
| 9 | EPPS | 0 |
| 10 | Tricine | 10.2 |
| 11 | Bicine | 17.64 |
| 12 | TAPS | 5.88 |
| 13 | CHES | 8.63 |
| 14 | CAPS | 17.26 |

As shown in FIG. 2 and Table 2, error color development seen in histamine-free samples was significantly suppressed in the experimental systems using HEPES, TAPSO, POPSO, HEPPSO, and EPPS buffers. BES, MOPS, TES, TAPS and CHES were also confirmed to have a tendency to inhibit error reaction.

### Example 3: Study on Buffer-2

EDTA (2Na), sodium bicarbonate/disodium carbonate, boric acid, and Tris (tris(hydroxymethyl)aminomethane) were all adjusted to 0.1 M and pH 9.0, and 0.4 mL of the resultant was added to a freeze-dried product of 0.2 mL of a reagent containing 2% trehalose, 1.08 mM NBT, and 41.5 μM 1-methoxy PMS. After mixing, the mixture was left at room temperature for 60 minutes, and the degree of color development was confirmed.

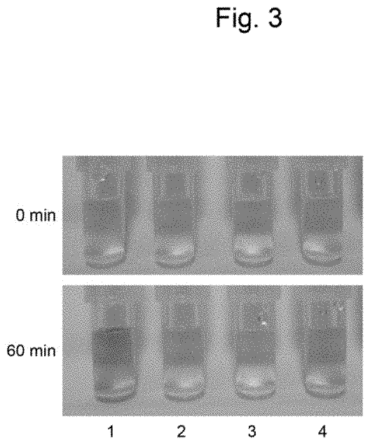

The results are shown in FIG. 3. For the purpose of also confirming the visual determination as shown in FIG. 3 with numerical data, colors were digitized in the same manner as in Example 1. For digitization, the “value of K” was used in all cases. The difference in color between before and after leaving the samples for 60 minutes is shown in Table 3 below.

TABLE 3

| Experimental | | Color |
|---|---|---|
| system No. | Buffer | change |
| 1 | EDTA | 5.49 |
| 2 | Carbonic acid | 0.4 |
| 3 | Boric acid | 0 |
| 4 | Tris | 0.78 |

As shown in FIG. 3 and Table 3, carbonic acid and boric acid exhibited an error reaction inhibitory effect.

### Example 4: Inhibition of Error Reaction in Presence of Sample-1

1 g of a fish meat sample (mackerel) finely minced using a kitchen knife was mixed with 1 ml of any of 1) a 0.1 M HEPPSO solution (pH 8.5), 2) a 0.1 M HEPPSO solution (pH 9.0), 3) a 0.1 M HEPPSO solution (pH 8.5) containing 25 mM boric acid, and 4) a 0.1 M HEPPSO solution (pH 9.0) containing 25 mM boric acid, thoroughly mixed using a spatula, and then stirred for 10 seconds in a vortex mixer for extraction. Each extracting liquid was collected using a cotton-tipped swab and the cotton-tipped swab was suspended in a polystyrene test tube containing 0.4 mL of any of the solutions 1) to 4) in advance, and the whole amount of the suspension was added to a freeze-dried product of 0.2 mL of a reagent containing 2% trehalose, 1.08 mM NBT, and 41.5 μM 1-methoxy PMS. After the reaction, the mixture was left at room temperature for 60 minutes, and the degree of color development was confirmed. As a blank control, the reagent was dissolved in 0.4 mL of a 0.1 M HEPPSO solution (pH 8.5 or pH 9.0). Incidentally, the fish meat sample used was a sample from which histamine was not detected by measuring the histamine content beforehand using Histamine Test (Japanese product name Check Color Histamine, manufactured by Kikkoman Biochemifa Company). In this experiment system, no histamine dehydrogenase was included in the reaction system. In other words, histamine was not contained in the fish meat sample, and furthermore, even if a minute amount of histamine was present in the reaction system, because histamine dehydrogenase was not included in the reaction system, it is believed that histamine does not (should not) bring about color development.

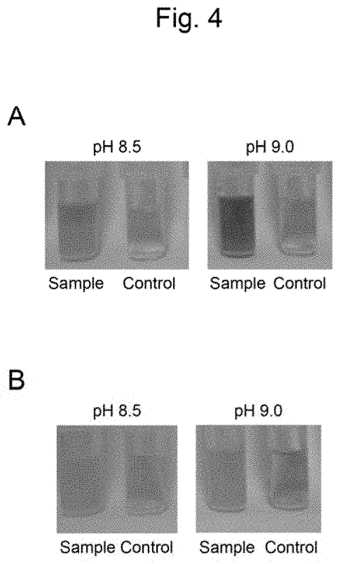

Results are shown in FIG. 4. As shown in FIG. 4A, coloring was observed even in the absence of histamine and histamine dehydrogenase. Therefore, even if HEPPSO suppressed reagent-derived error reaction, when a sample such as fish meat was further added into the reaction system, it (HEPPSO) could not completely suppress color development considered as error reaction not derived from histamine. On the other hand, as shown in FIG. 4B, by further adding boric acid to HEPPSO, even when a sample such as fish meat was added, color development believed to be an error reaction not derived from histamine could be significantly suppressed (inhibited).

For the purpose of also confirming the visual determination as shown in FIG. 4 with numerical data, colors were digitized in the same manner as in Example 1. For digitization, the “value of K” was used in all cases. The difference in color between before and after leaving the samples for 60 minutes is shown in Table 4 below.

TABLE 4

| | | Color change |
|---|---|---|
| 25 mM boric acid | Sample | 0.39 |
| 0.1M HEPPSO pH 8.5 | Control | 0.39 |
| 25 mM boric acid | Sample | 2.75 |
| 0.1M HEPPSO pH 9 | Control | 0.39 |
| No boric acid | Sample | 4.7 |
| 0.1M HEPPSO pH 8.5 | Control | 1.18 |
| No boric acid | Sample | 39.22 |
| 0.1M HEPPSO pH 9 | Control | 4.71 |

As described above, error color development was seen at both pH 8.5 and 9.0. Even color development as large as that corresponding to approximately 50 ppm color development using a histamine standard was observed, indicating that error color development may cause misjudgment in histamine detection. This also indicated that error color development can be suppressed by the addition of boric acid.

### Example 5: Inhibition of Error Reaction in Presence or Sample-2

1 g of a minced fish meat sample (mackerel) was subjected to extraction with 1 mL of 0.1 M HEPPSO (PH 8.5) containing 1) a control or 25 mM or lower boronic acid, 2) phenylboronic acid, 3) 4-chlorophenylboronic acid, 4) 4-fluorophenylboronic acid, 5) butylboronic acid, or 6) 3-[(tert-buloxycarbonyl)amino]phenylboronic acid. For the extraction, the fish meat was thoroughly mixed with each extracting liquid using a spatula, and then stirred for 10 seconds in a vortex mixer. The extracting liquid was collected using a cotton-tipped swab and the cotton-tipped swab was suspended in a polystyrene test tube containing 0.4 mL of any of the solutions 1) to 6) in advance, and the whole amount of the suspension was added to a freeze-dried product of 0.2 mL of a reagent containing 2% trehalose, 1.08 mM NBT, and 41.5 μM 1-methoxy PMS. After the reaction, the mixture was left at room temperature for 60 minutes, and the degree of color development was confirmed.

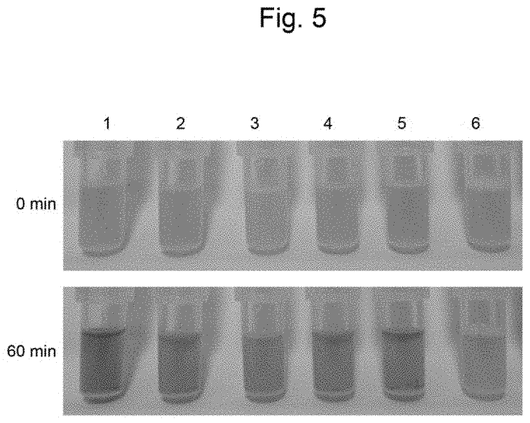

The results are shown in FIG. 5. For the purpose of also confirming the visual determination as shown in FIG. 5 with numerical data, colors were digitized in the same manner as in Example 1. For digitization, the “value of K” was used in all cases. The difference in color between before and after leaving the samples for 60 minutes is shown in Table 5 below.

TABLE 5

| Experimental | | Color |
|---|---|---|
| system No. | Adduct | change |
| 1 | Control | 15.69 |
| 2 | Phenylboronic acid | 8.23 |
| 3 | 4-Chlorophenylboronic acid | 8.63 |
| 4 | 4-Fluorophenylboronic acid | 7.46 |
| 5 | Butylboronic acid | 7.45 |
| 6 | 3-[(tert-Butoxycarbonyl)amino] | 1.57 |
| | phenylboronic acid | |

As shown in FIG. 5 and Table 5, the addition of boronic acid was able to strongly suppress sample-derived error reaction. This effect was significant, particularly, when 3-[(tert-butoxycarbonyl)amino]phenylboronic acid was added.

### Example 6: Study on Boric Acid Concentration

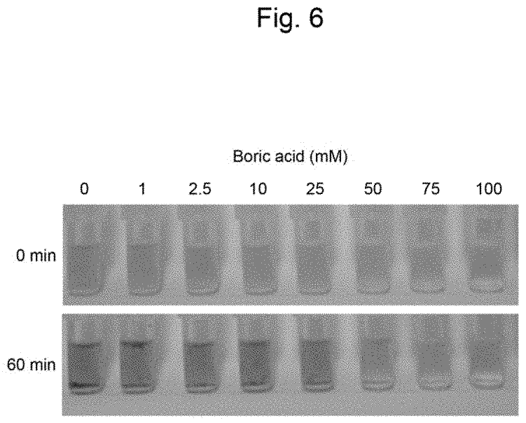

2 g of a minced fish meat sample (mackerel) was subjected to extraction in the same manner as in Example 5 using 2 mL of 0.1 M HEPPSO (pH 8.5) containing boric acid having each concentration from 0 mM to 100 mM. After the extraction, a cotton-tipped swab was suspended in 0.4 mL of 0.1 M HEPPSO (pH 8.5) containing boric acid having each concentration, and the whole amount of the suspension was added to a freeze-dried product of 0.2 mL of a reagent containing 2% trehalose, 1.08 mM NBT, and 41.5 μM 1-methoxy PMS. After the reaction, the mixture was left at room temperature for 60 minutes, and the degree of color development was confirmed. The results are shown in FIG. 6.

For the purpose of also confirming the visual determination as shown in FIG. 6 with numerical data, colors were digitized in the same manner as in Example 1. For digitization, the “value of K” was used in all cases. The difference in color between before and after leaving the samples for 60 minutes is shown in Table 6 below.

TABLE 6

| Boric acid | Color |
|---|---|
| concentration (mM) | change |
| 0 | 7.84 |
| 1 | 5.88 |
| 2.5 | 5.49 |
| 10 | 5.1 |
| 25 | 3.92 |
| 50 | 0.78 |
| 75 | 0.78 |
| 100 | 0.79 |

As described above, boric acid reduced error reaction in a concentration-dependent manner.

### Example 7: Inhibition of Error Reaction by SDS

2 g of a minced fish meat sample (mackerel) was subjected to extraction in the same manner as in Example 5 using 2 mL of 0.1 M HEPPSO (PH 8.0) each containing SDS at a concentration of from 0% to 1%. After the extraction, a cotton-tipped swab was suspended in 0.4 mL of 0.1 M HEPPSO (pH 8.5) containing SDS at each concentration, and the whole amount of the suspension was added to a freeze-dried product of 0.2 mL of a reagent containing 2% trehalose, 1.08 mM NBT, and 41.5 μM 1-methoxy PMS. After the reaction, the mixture was left at room temperature for 60 minutes, and the degree of color development was confirmed.

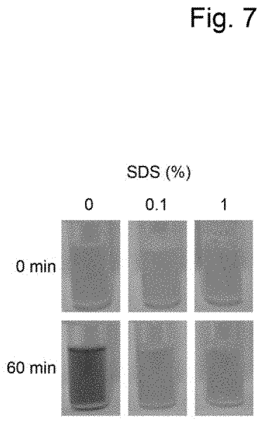

The results are shown in FIG. 7. For the purpose of also confirming the visual determination as shown in FIG. 7 with numerical data, colors were digitized in the same manner as in Example 1. For digitization, the “value of K” was used in all cases. The difference in color between before and after leaving the samples for 60 minutes is shown in Table 7 below.

TABLE 7

| SDS | |
|---|---|
| concentrabon | Color change |
| 0 | 23.53 |
| 0.1 | 1.17 |
| 1 | 1.57 |

As described above, SDS was also shown to reduce error reaction.

### Example 8: Confirmation that Boric Acid does Not Inhibit Color Reaction

2 g of a minced fish meat sample (mackerel) was subjected to extraction in the same manner as in Example 5 using 2 mL of 0.1 M HEPPSO (pH 9.0) containing 25 mM or 100 mM boric acid. After the extraction, 0, 10, 25, 50, 75, or 100 ppm histamine was added to 0.4 mL of 0.1 M HEPPSO (pH 9.0) containing boric acid having each concentration. A cotton-tipped swab was suspended in this solution, and the whole amount of the suspension was added to a freeze-dried product of 0.2 mL of a reagent containing 2% trehalose, 1.08 mM NBT, and 41.5 μM 1-methoxy PMS. After the reaction, the mixture was left at room temperature for 15 minutes, and the degree of color development was confirmed.

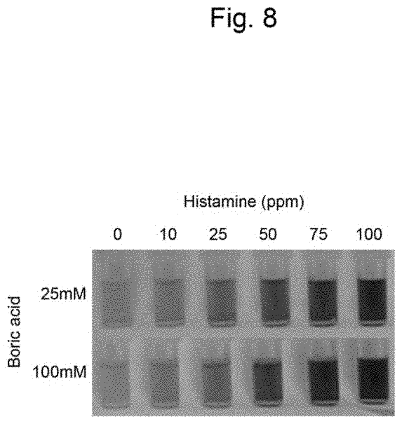

The results are shown in FIG. 8. For the purpose of also confirming the visual determination as shown in FIG. 8 with numerical data, colors were digitized in the same manner as in Example 1. For digitization, the “value of K” was used in all cases. The difference in color between before and after leaving the samples for 15 minutes is shown in Table 8 below.

TABLE 8

| Histamine | Color change | |
|---|---|---|
| concentration | 25 mM | 100 mM |
| (ppm) | Boric acid | Boric acid |
| 0 | 5.88 | 2.75 |
| 10 | 9.41 | 8.24 |
| 25 | 19.61 | 18.04 |
| 50 | 35.69 | 39.61 |
| 75 | 45.49 | 52.94 |
| 100 | 57.25 | 63.53 |

As shown in FIG. 8 and Table 8, measurement was achieved even at a boric acid concentration of 100 mM, indicating that the addition of boric acid has no adverse effect on color development derived from histamine.

### Example 9: Preparation of a Histamine Calibration Curve by Cyclic Voltammetry (Fish Meat-Non-Supplemented System)

0.2 mL of a reagent containing 2% trehalose, 1.08 mM NBT, 41.5 μM 1-methoxy PMS and 0.128 U dehydrogenase was freeze-dried and then dissolved by the addition of 500 μL of a 0.1 M boric acid solution (PH 8.5). For comparison, a solution was similarly prepared using a 0.1 M EDTA solution used as a solution for extraction in Histamine Test (manufactured by Kikkoman Biochemifa Company), instead of the 0.1 M boric acid solution (pH 8.5). 20 μL each of these solutions was added dropwise to printed electrodes to perform cyclic voltammetry. The printed electrodes used were SCREEN-PRINTED ELECTRODES (manufactured by Metrohm DropSens, DRP-110) in which a carbon working electrode (12.6 mm²) and a silver reference electrode were printed, and connected to ALS electrochemical analyzer 814D (manufactured by BAS Inc.) via a dedicated connector (manufactured by Metrohm DropSens, DRP-CAC). Further, 1 or 2 μL of a 1000 ppm histamine solution was added thereto, and cyclic voltammetry was performed in a similar manner for each solution. The potential was swept at 20 mV per second. Oxidation current values at −150 mV (vs. Ag/Ag⁺) were plotted to determine the relationship between histamine concentrations and current values.

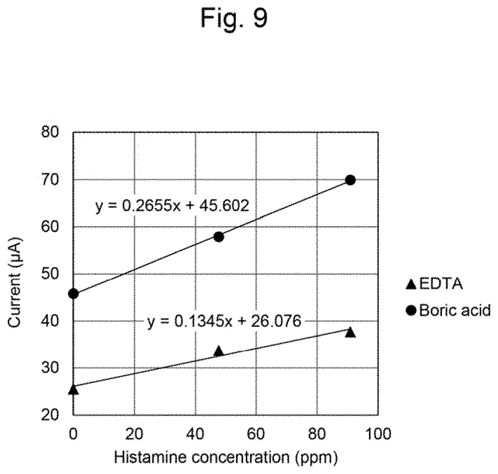

As a result, as shown in FIG. 9, both the boric acid solution and the EDTA solution produced favorable linear histamine calibration curves. When boric acid was used in the solution, a larger current value was obtained, the slope was larger, and the S/N ratio was slightly improved, as compared with the case of EDTA. These results indicated that use of a boric acid solution improves the accuracy of histamine detection by cyclic voltammetry.

### Example 10: Preparation of a Histamine Calibration Curve by Chronoamperometry (in the Presence of Fish Meat Sample)

2 g of minced mackerel was subjected to extraction with 2 mL of water in the same manner as in Example 5. 0.2 mL of a reagent containing 2% trehalose, 41.5 μM 1-methoxy PMS and 0.128 U histamine dehydrogenase was freeze-dried and then, 30 μL of a 0.1 M HEPPSO solution (pH 8.5) or a 0.1 M HEPPSO/0.1 M boric acid solution (pH 8.5), 10 μL of the extracting liquid of the fish meat, and 10 μL of a 3 M NaCl solution were each added dropwise to printed electrodes to perform chronoamperometry. The printed electrodes used were SCREEN-PRINTED ELECTRODES (manufactured by Metrohm DropSens, DRP-110) in which a carbon working electrode (12.6 mm²) and a silver reference electrode were printed, and connected to ALS electrochemical analyzer 814D (manufactured by BAS Inc.) via a dedicated connector (manufactured by Metrohm DropSens, DRP-CAC). Further, 1, 2 or 4 μL of a 1000 ppm histamine solution was added thereto, and chronoamperometry was performed in a similar manner. For measurement, a potential of +200 mV (vs. Ag/AgCl) was applied. Current values after a lapse of 10 seconds from the start of measurement were plotted to determine the relationship between histamine concentrations and current values.

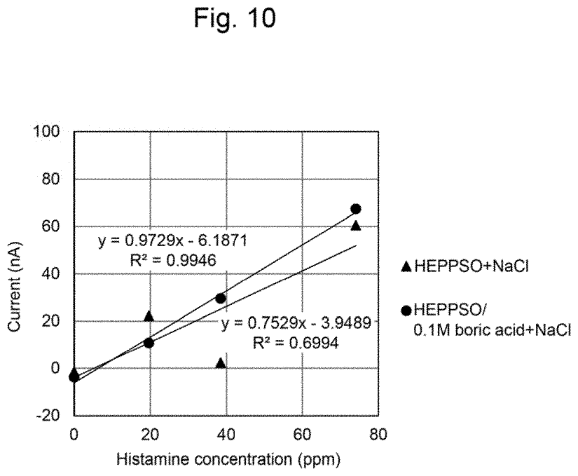

As a result, as shown in FIG. 10, the correlation between histamine concentrations and current values tended to be difficult to obtain without the addition of boric acid in the presence of the fish meat sample (correlation coefficient R²=0.6994). The addition of boric acid suppressed the influence of the fish meat sample and produced a favorable correlation (correlation coefficient R²=0.9946). These results indicated that use of a boric acid solution improved the accuracy of histamine detection by chronoamperometry.

### INDUSTRIAL APPLICABILITY

According to the present invention, error reaction not derived from enzyme reaction can be reduced in methods for measuring histamine using histamine dehydrogenase. As a result, histamine can be detected, particularly, even in low-concentration ranges.

All publications, patents and patent applications cited herein are incorporated herein by reference in their entirety.

1. A kit for detecting histamine, comprising
(i) histamine dehydrogenase, and
(ii-a) boric acid or a salt thereof, and/or a boronic acid represented by the following formula (I) or (II): 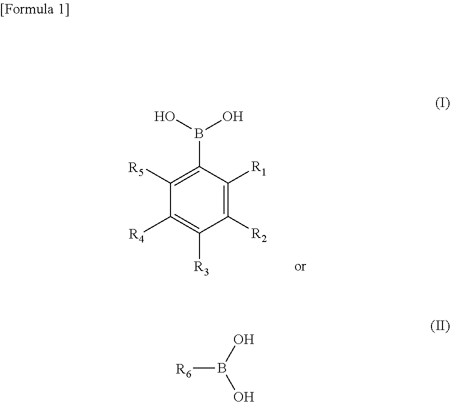
wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

2. An electrochemical sensor capable of detecting the oxidation of histamine by histamine dehydrogenase, comprising
(i) histamine dehydrogenase, and
(ii-a) boric acid or a salt thereof, and/or a boronic acid represented by the following formula (I) or (II): 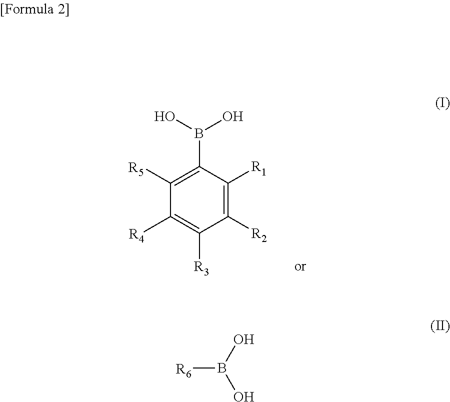
wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate.

3. The kit according to claim 1, further comprising a mediator.

4. The kit according to claim 3, wherein the mediator is selected from the group consisting of 1-methoxy PMS (1-methoxy-5-ethylphenazinium ethyl sulfate), PMS (phenazinium methyl sulfate), PES (phenazinium ethyl sulfate) and 1-methoxy PES (1-methoxy-5-ethylphenazinium ethyl sulfate).

5. The kit according to claim 1, further comprising a coloring reagent that develops a color when histamine is oxidized by the histamine dehydrogenase.

6. The kit according to claim 5, wherein the coloring reagent is tetrazolium salt.

7. The kit according to claim 1, further comprising a buffer comprising a compound having a zwitterion and having no carboxy group, the buffer being selected from the group consisting of BES, MOPS, TES, HEPES, EPPS, TAPS, CHES, CAPS, TAPSO, POPSO, HEPPSO, ACES, Bis-Tris, MES, MOPSO, and PIPES, or Tris (tris(hydroxymethyl)aminomethane) or a carbonate buffer.

8. The kit according to claim 7, wherein the buffer is a buffer comprising a compound having a zwitterion and having a sulfo group, the buffer being selected from the group consisting of BES, MOPS, TES, HEPES, EPPS, TAPS, CUES, CAPS, TAPSO, POPSO, HEPPSO, ACES, MES, MOPSO, and PIPES.

9. The kit according to claim 8, wherein the buffer is a buffer comprising a compound having a zwitterion, having a sulfo group, and having a hydroxy group at position 2, the buffer being selected from the group consisting of TAPSO, POPSO, HEPPSO, and MOPSO.

10. The kit according to claim 1, wherein the kit comprises the boric acid or the salt thereof, and/or the boronic acid or the salt thereof such that a final concentration of said boric acid or the salt thereof, and/or the boronic acid or the salt thereof at the time of measurement is 120 mM or lower.

11. The kit according to claim 1, wherein the alkyl sulfate is sodium lauryl sulfate.

12. The kit according to claim 1, wherein
the kit comprises a sample collection part and a reaction part, and
the reaction part comprises the histamine dehydrogenase and the boric acid or the salt thereof, and/or the boronic acid or the salt thereof.

13. A method for detecting histamine, comprising:
(A) using (i) histamine dehydrogenase, and (ii-a) boric acid or a salt thereof, and/or a boronic acid represented by the following formula (I) or (II): 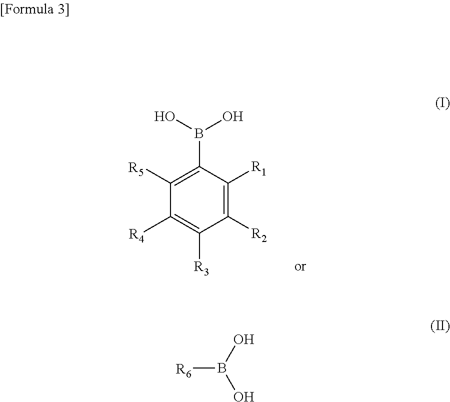
wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(ii-b) alkyl sulfate, or
(B) using an electrochemical sensor capable of detecting the oxidation of histamine by histamine dehydrogenase, wherein the sensor comprises
(iii) histamine dehydrogenase, and
(iv-a) boric acid or a salt thereof, and/or a boronic acid represented by the following formula (I) or (II): 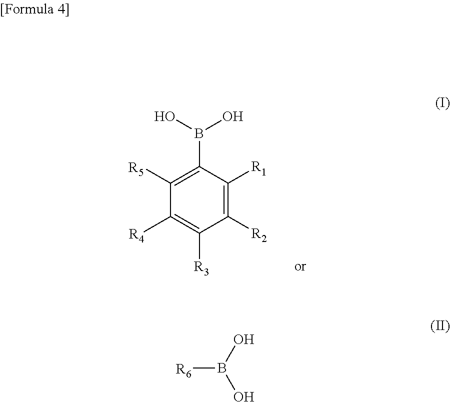
wherein R₁ to R₅ are each independently selected from the group consisting of H, a boronyl group, a halogen group, a hydroxy group, a carboxy group, a nitro group, an amino group, a sulfo group, a thiol group, a tert-butoxycarbonylamino group, and a substituted or unsubstituted C₁ to C₁₀ alkyl group, alkenyl group, or alkynyl group, and
R₆ is selected from the group consisting of a C₁ to C₁₀ alkyl group, alkenyl group, and alkynyl group substituted with at least one substituent selected from the group consisting of a boronyl group, a halogen group, a hydroxy group, a carboxy group, a nitro group, an amino group, a sulfo group and a thiol group, or an unsubstituted C₁ to C₁₀ alkyl group, alkenyl group, and alkynyl group
or a salt thereof, and/or
(iv-b) alkyl sulfate.

14. (canceled)

15. The method according to claim 13, further comprising using a mediator.

16. The method according to claim 15, wherein the mediator is selected from the group consisting of 1-methoxy PMS, PMS, PES and 1-methoxy PES.

17. The method according to claim 13, further comprising using a coloring reagent that develops a color when histamine is oxidized by the histamine dehydrogenase.

18. The method according to claim 17, wherein the coloring reagent is tetrazolium salt.

19. The method according to claim 13, further comprising using a buffer comprising a compound having a zwitterion and having no carboxy group, the buffer being selected from the group consisting of BES, MOPS, TES, HEPES, EPPS, TAPS, CHES, CAPS, TAPSO, POPSO, HEPPSO, ACES, Bis-Tris, MES, MOPSO, and PIPES, or Tris or a carbonate buffer.

20-21. (canceled)

22. The method according to claim 13, wherein the boric acid or the salt thereof, and/or the boronic acid or the salt thereof is used at a final concentration of 120 mM or lower at the time of measurement.

23. The method according to claim 13, wherein the alkyl sulfate is sodium lauryl sulfate.
